(19) United States
(12) Patent Application Publication
(10) Pub. No.: US 2023/0301578 A1
(43) Pub. Date: Sep. 28, 2023

(54) Device And Method Of Monitoring Mental State And Jaw Movements

(71) Applicants: Anhui Huami Health Technology Co., Ltd., Hefei (CN); Zepp, Inc., Cupertino, CA (US)

(72) Inventors: Sam Rahbar, Vancouver (CA); Artem Galeev, Vancouver (CA)

(21) Appl. No.: 17/703,612

(22) Filed: Mar. 24, 2022

(51) Int. Cl. A61B 5/375; A61B 5/00; A61B 5/372; A61B 5/16

(52) U.S. Cl. CPC A61B 5/375; A61B 5/6817; A61B 5/721; A61B 5/372; A61B 5/165; A61B 5/7264

(57) ABSTRACT

A device including: one or more ear loop, three or more electroencephalogram sensors (EEG sensors), and one or more movement sensors. The one or more ear loops configured to extend at least partially around an ear of a user. The one or more ear loops include: the three or more EEG sensors. The three or more EEG sensors are distributed along the one or more ear loops so that the three or more EEG sensors extend around the ear of the user. A support post extends from the one or more ear loops. An ear tip is connected to and supported by the post. The one or more movement sensors are configured to monitor movement of the user. The one or more movement sensors include one or more pizeoelectric sensors (PES).

### CROSS REFERENCES TO RELATED APPLICATION(S)

NONE.

### FIELD

The teachings herein relate to a device and method of non-invasively monitoring a mental state of a user such as a stress of the user.

### BACKGROUND

As cell phones and other wireless devices continue to increase in use and users are increasing use of hearing devices and microphones to access their cell phones wirelessly. Moreover, many states are requiring hands free use of phones while driving, further increasing the use of headphones and related devices. These devices are located on a user in and around an ear of a user and users may keep these devices in use continuously throughout the day.

It would be attractive to have a device and method for detecting stress changes of a user in real time. What is needed is a device that monitors brain waves of a user. It would be attractive to have a device, method, or both of tracking motions of an ear, head, jaw, neck, or a combination thereof to determine patterns, receptiveness, indications of changes in stress, or a combination thereof. What is needed is a device that filters macro movements out of a brain wave signal from an EEG sensors so that the brain wave signal accurately reflects a mental state of the user.

### SUMMARY

The present teachings provide: a device including: one or more ear loop, three or more electroencephalogram sensors (EEG sensors), and one or more movement sensors. The one or more ear loops configured to extend at least partially around an ear of a user. The one or more ear loops include: the three or more EEG sensors. The three or more EEG sensors are distributed along the one or more ear loops so that the three or more EEG sensors extend around the ear of the user. A support post extends from the one or more ear loops. An ear tip is connected to and supported by the post. The one or more movement sensors are configured to monitor movement of the user. The one or more movement sensors include one or more pizeoelectric sensors (PES).

The present teachings provide: a device including: two or more sensors and a processor.

The two or more sensors include: two or more electroencephalogram sensors (EEG sensors) and one or more movement sensors. The two or more EEG sensors are configured to monitor brain activity of a user. The one or more movement sensors are configured to monitor movement of the user, wherein the one or more movement sensors include one or more piezoelectric sensors (PES). The processor is configured to: detect the brain activity of the user with the two or more EEG sensors; detect movements of the user with the one or more PES to generate movement data; isolate the brain activity by excluding the movement data from the brain activity to generate filtered data, and provide feedback to the user regarding a current mental state of the user based upon the filtered data.

The present teachings provide: a method including: monitoring brain activity and movements of a user to determine a current mental state of the user. The monitoring of brain activity of the user is performed with two or more electroencephalogram sensors (EEG sensors) that are spaced apart and located adjacent to an ear of the user. Generating brain activity data from the monitored brain activity. The monitoring of the movements of the user is performed with one or more movement sensors that include one or more piezoelectric sensors (PES). Generating movement data from the monitored movements of the user. Classifying a current mental state of the user based on the EEG sensors. Providing a personalized score to the user based on the current mental state.

The present teachings provide: a device and method for detecting stress changes of a user in real time. The present teachings provide a device that monitors brain waves of a user. The present teachings provide a device, method, or both of tracking motions of an ear, head, jaw, or a combination thereof to determine patterns, receptiveness, indications of changes in stress, or a combination thereof. The present teachings provide a device that filters macro movements out of a brain wave signal from an Electroencephalogram sensor (EEG Sensor) so that the brain wave signal accurately reflects a mental state of the user.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A depicts a device in use by a user.

FIG. 1B depicts a device.

FIG. 1C is a cross-sectional view of the device of FIG. 1B

FIG. 1D is a cross-sectional view of a device.

FIG. 1E is a cross-sectional view of a device.

FIG. 2 depicts a flow diagram of the teachings herein.

### DETAILED DESCRIPTION

Example implementations will be described in detail here, and examples thereof are shown in the accompanying drawings. When the following description refers to the accompanying drawings, unless otherwise indicated, the same numerals in different accompanying drawings indicate the same or similar elements. Implementations described in the following example implementations do not represent all implementations consistent with the present application. On the contrary, they are merely examples of devices consistent with some aspects of the present application as detailed in the appended claims.

Terms used in the present application are only for the purpose of describing specific implementations, and are not intended to limit the present application. Unless otherwise defined, technical or scientific terms used in the present application shall have general meanings understood by those of general skills in the art to which the present application belongs. Similar words such as “one” or “a” used in the specification and claims of the present application do not mean a quantity limit, but mean that there is at least one. Similar words such as “including” and “containing” mean that an element or item before “including” or “containing” covers an element, item, or its equivalent listed after “including” or “containing,” and does not exclude other elements or items. Similar words such as “connect” or “connected” are not limited to physical or mechanical connections, and may include electrical connections, whether direct or indirect. “Multiple” includes two and is equivalent to at least two. Singular forms of “a,” “said,” and “the” used in the specification and appended claims of the present application are also intended to include plural forms, unless the context clearly indicates other meanings. It should also be understood that the term “and/or” as used herein refers to and includes any or all possible combinations of one or more associated listed items.

Disclosed herein is a device that senses, measures, analyzes, displays physiological information, or a combination thereof. The physiological information may include monitoring, identify, or both stress, a mental status, stress related movements, or a combination thereof. In one aspect, the device may be a wearable device comprising an ear device, a movement sensor, an electroencephalogram sensor (EEG sensor), a pizeoelectric sensor (PES), or a combination thereof. The wearable device may be worn on a user's body such that one or more sensors of the upper and lower modules contact a targeted area of tissue. In one implementation, the wearable device is an ear bud, an ear device, an over the ear device, insertable into an ear canal, or a combination thereof.

The present teachings provide a wearable device that functions to monitor physiological characteristics (e.g., stress, repetitive motions, jaw activity) of a user. The wearable device may monitor the physiological characteristics (e.g., brain activity) of a user at a first location or within a first region. The wearable device may monitor two regions. For example, one wearable device may be located on a first ear and a second wearable device may be located on a second ear. The wearable device may monitor a first location and a second location simultaneously for physiological characteristics (e.g., brain activity and movement). For example, the wearable device may have a first portion (e.g., an ear tip) that extends into an ear canal of a user and a second portion (e.g., an ear loop) that extends around an ear of a user.

The wearable device may be an ear device. The wearable device may include two ear devices. The ear devices may be a left ear device and a right ear device. The ear devices may be identical to one another. The ear devices as discussed herein may recite one; however, as taught herein that may mean that the wearable device includes two ear devices that are used in tandem since there may be a device in each ear. However, the wearable device may only include one ear device or sensors in one of the ear devices. The ear device may fully extend into an ear, be free of extension into an ear canal, extend around an ear, or a combination thereof. The ear device may operate when fully inserted, partially inserted, or completely withdrawn. The ear device may operate when in communication with an ear of a user. The ear device may include one or more components that maintain the ear device in contact with a user. The ear device may include an ear loop, an ear tip, a support post, or a combination thereof.

The one or more ear loops may function to extend around an ear, support the ear device, support sensors, or a combination thereof. The ear loop may extend from a central region of an ear over a top of the ear and down a rear side of the ear. The ear loop may extend from substantially a top of the ear to substantially a bottom of the ear. The ear loop may prevent the ear device from falling from an ear during movement of the user. The ear loop may be made of or include plastic, metal, rubber, an elastomer, polycarbonate (PC), acrylic, polymethyl methacrylate (PMMA), polyethylene (PE), polypropylene (PP), polyethylene terephthalate (PET), polyvinyl chloride (PVC), acrylonitrile-butadiene-styrene (ABS), or a combination thereof. The ear loop may be flexible. For example, the ear loop may be contoured to a shape of an ear of a user. The ear loop may flex to grip a portion of an ear. The ear loop may be rigid. The ear loop may be “C”-shaped, “U”-shaped, “T”-shaped, or a combination thereof. The ear loop may be connected to a speaker, a microphone, a support post, an ear tip, or a combination thereof.

The one or more ear tips function to provide sound to a user, receive sounds from the user, connect the ear device to the user, or a combination thereof. The ear tip may extend into an ear canal, The ear tip may cover an ear canal. The ear tip may provide sound to a user. The ear tip may include one or more speakers. The ear tip may be movable. The ear tip may be movable laterally, axially, longitudinally, rotationally, or a combination thereof. The ear tip may be compressible. The ear tip may be rigid. A portion of the ear tip may be rigid, and a portion of the ear tip may be flexible. The ear tip may include one or more speakers. The ear tip may locate the one or more speakers proximate to an ear canal of a user. The ear tip may extend cantilever from the ear loop, a support post, or both.

The support post functions to connect the ear loop and the ear tip, to support the ear tip within and/or proximate to an ear canal, to provide a flexible member, or a combination thereof. The support post may be rigid, movable, have a rigid portion, have a movable portion, be solid, hollow, or a combination thereof. The support post may have a microphone on one end and a speaker on another end. The support post may include one or more pieces, two or more pieces, or three or more pieces. The support post may be straight, curved, or both. The support post may extend axially away from an ear canal. The support post may extend around an ear (e.g., have a “C”-shape). The support post may include one or more pieces, two or more pieces, or three or more pieces.

The one or more support post may include two pieces that are movable relative to one another. The support posts may have one post that is static (e.g., a primary support post) and one post that is movable (e.g., secondary support post). The primary support post may connect directly to the ear loop. The primary support post may have a first cross-sectional thickness (e.g., diameter). The secondary support post may have a second cross-sectional thickness (e.g., diameter). The first cross-sectional thickness may be larger than the second cross-sectional thickness. The first cross-sectional thickness may be smaller than the second cross-sectional thickness. The primary support post may receive all or a portion of the secondary support post. The primary support post may be rigid and the secondary support post may be movable within and/or around the primary support post. The primary support post and the secondary support post may be mono stable relative to one another. For example, a bias member may be located between the primary support post and the secondary support post. The bias member may axially move the primary support member and the primary support member to a static state or a resting state.

The one or more bias members functions to move the support post to a static position, a steady state, an unbiased position, or a combination thereof. The one or more bias members may be compressible, expandable, or both. The bias members may be compressed by a user so that the compression is applied to a sensor. The one or more bias members may be made of or include rubber, an elastomer, spring steel, stainless steel, a ferrous based metal, or a combination thereof. One or more wires may extend through or along the one or more bias members to provide power, signals, or both.

The power source functions to power the ear device, the sensors, or both. The power source may be provided through wires, electrical connectors, or both. The power source may be an internal power source, a battery, an external power source, a cell phone, a computer, or a combination thereof. The power source may be an AC power source or a DC power source. The power source may be a wire or wires that also transmits data or signal therethrough. The power source may power one or more sensors or two or more sensors of the ear device.

The sensors function to monitor physiological characteristics of a user. The sensor may include one or more of a movement sensor, an electroencephalogram sensor, or both. The sensors may be located entirely within the ear device, partially within and partially outside the ear device, completely outside of the ear device, or a combination thereof. The sensors may be located within the ear tip, the support posts, the ear loop, or a combination thereof. The sensors may be spaced apart so that one sensor is located on or within the ear loop and one sensor is located on or within the ear tip and/or the support post. Each ear device (e.g., left side and right side) may include each of the sensors taught herein (e.g., one or more or two or more sensors).

The sensors may include a one or more electroencephalogram sensors (e.g., an EEG sensor). The EEG sensors function to monitor brain waves, electrical activity, brain activity, or a combination thereof (hereinafter brain activity). The EEG sensors may monitor brain activity relative to a reference location. The EEG sensors may be two or more EEG sensors, three or more EEG sensors, or four or more EEG sensors. The EEG sensors may be a multitude of EEG sensors that are spaced apart and located proximate to a head of a user. The EEG sensors may be spacedly located on an ear loop and located proximate to a head of a user, behind an ear of the user, or both (e.g., an EEG sensor may be located at a top, bottom, middle, or a combination of an ear loop). The multiple EEG sensors may monitor brain activity simultaneously. The brain activity may be monitored by multiple EEG sensors and the multiple EEG sensors may be used in tandem to determine the brain activity of the user. Each EEG sensor may provide a waveform of brain activity. The EEG sensors may be used in conjunction with one or more movement sensors.

The one or more movement sensors function to monitor movement of the user, movements of the user proximate to the EEG sensors, movement of a user's head, movement of the user's jaw, movement of the user's neck, or a combination thereof. The one or more movement sensors may be or include piezoelectric sensors (PES), accelerometer, ultrasound, gyroscope, light sensors, optic cables, or a combination thereof. The one or more movement sensors may indirectly monitor movement. For example, the ear tip, the support post, or both may be moved and the ear tip, the support post, or both may contact the movement sensors to determine movement of the user. The movement sensors may directly move. For example, a portion of a movement sensor may be in direct contact with the user so that movement of the user moves the movement sensor. The movement sensor may pass a signal through a wall of the ear device. For example, an ultrasound wave may pass through the ear device into contact with a user. A signal may pass from an outside of the ear device into contact with the user. For example, if a light sensor is used light may be passed into contact with the user. At least one of the movement sensors may be a PES.

The PES functions to detect movements of a user. The PES may detect movements of a head, jaw, neck, ear, or a combination thereof. The PES may detect movement by detecting movements of the ear tip, the ear tip relative to the ear loop, the primary support post relative to the secondary support post, or a combination thereof. The PES may be compressible, flexible, bent, moved, vibrated, or a combination thereof. The PES may detect movement by being indirectly contacted. For example, the ear tip may move relative to the support post and this movement may be detected. The PES may detect movement by direct contact. For example, all or a portion of the PES may be located outside of the ear device and may be contacted by movements of the user so that the PES is deformed and the movements are detected. The PES may detect movement, micro-movements, macro-movements, or a combination thereof. The PES may be able to distinguish between types of movements. The PES may output a movement data. The movement data may be filtered to categorize the movement data based upon types of movements. The movement data may be overlaid with brain activity, charted with brain activity in a time dependent manner, or both. For example, the brain activity and the movement activity may be charted simultaneously so that movements that affect brain activity (e.g., outliers) may be filtered, removed, or both.

The movement sensors may include an accelerometer, gyroscope, or both. The accelerometer or gyroscope functions to measure movement of a user via acceleration or velocity changes respectively. The accelerometer or gyroscope may measure micro-movements, macro-movements, or both. The accelerometer or gyroscope, may measure respiration, heartbeats, facial movements, jaw movements, head movements, neck movements, or a combination thereof. The accelerometer, gyroscope, or both may be located in or in contact with the ear tip, support post, ear loop, or a combination thereof. The accelerometer, gyroscope, or both may be used in conjunction with the PES. The movement sensors may monitor micro-movements, acceleration, velocity changes, or a combination thereof.

The ear device may include other sensors. The sensors may monitor electrical signals of the heart, micro-movements, or both. For example, the heart may receive electrical signals that cause the heart to beat and the sensors may monitor the electrical signals. The sensors may monitor pulse signals. The pulse signals may be movement of a part of the body caused by blood being moved through the body. The pulse signals may be movement of blood through a vein or artery. The pulse signals may monitor expansion and contraction of veins and arteries using a light. The sensors may include an optical sensor (e.g., PPG), a pulse pressure sensor (PP), a pressure sensor, an electrocardiogram (ECG), or a combination thereof. The sensors may be any device that may measure a heart rate. The optical sensors may provide a heart rate via a PPG. The sensor may provide a heart rate via a pressure sensor (PP) or ECG. The sensors may be used in conjunction with the EEG sensors. For example, if the brain activity is indicative of stress then the heart rate or blood pressure of the user may increase and cross-reference of the data from the sensors may indicate that the measurements are accurate.

The ear device may comprise one or more sensors, including but not limited to optical sensors (e.g., PPG), electrocardiogram (ECG) sensors/electrodes, bio impedance sensors, galvanic skin response sensors, tonometry/contact sensors, accelerometers, gyroscopes, pressure sensors, acoustic sensors, electro-mechanical movement sensors, or electromagnetic sensors, or a combination thereof. The one or more optical sensors (e.g., PPG) may comprise one or more light sources for emitting light proximate a targeted area of tissue and one or more optical detectors for detecting either reflected light (where an optical detector is located on the same side of the targeted area as the light source(s), i.e., within the same module) or transmitted light (where an optical detector is located opposite the light source(s), i.e., within an opposing module). The optical sensor may be a light emitting diode and photodiode (e.g., LED+photodiode) to measure PPG. The pressure sensor may monitor pulse pressure to provide a PP reading.

While the systems and devices described herein may be depicted as ear device, one skilled in the art will appreciate that the systems and methods described below can be implemented in other contexts, including the sensing, measuring, analyzing, and display of physiological data gathered from a device worn at any suitable portion of a user's body, including but not limited to, other portions of the head, other extremities, the face, the forehead, neck, or a combination thereof. For example, the sensors taught herein may be located within a headband.

The physiological characteristics sensed, measured, analyzed, or displayed can include but is not limited to heart rate information, ECG waveforms, calorie expenditure, step count, speed, blood pressure, oxygen levels, pulse signal features, cardiac output, stroke volume, respiration rate of a user, breathing movements, heart rate of a user, a respiratory signal, mental status, repetitive movements, repetitive activities, jaw movement, eating patterns, stress level, or a combination thereof. The physiological characteristics may be converted into physiological information, identify a type of pattern currently being monitored, or both. The physiological information may be any information associated with a physiological characteristic derived directly or indirectly from information received by one or more sensors of the wearable device. The physiological characteristics may be used in the context of, for example, health and wellness monitoring, athletic training, physical rehabilitation, patient monitoring, anxiety monitoring, mental status monitoring, asthma monitoring, or a combination thereof. The physiological characteristics including but not limited to mental health, brain activity, physical characteristics indicative of mental health, stress, anxiety, guilt, fear, jealousy, overwhelmed emotions, jaw movements, neck movements, or a combination thereof.

The processor functions to detect brain activity, analyze brain activity, EEG sensor data, movements, movement data, or a combination thereof. The processor may monitor brain activity from one or more, two or more, three or more, four or more, five or more, or six or more EEG sensors. The processor may monitor or detect movement activity from one or more, two or more, three or more, four or more, five or more, or six or more movement sensors. The processor may convert brain activity into brain activity data. The processor may isolate the brain activity or brain activity data by removing movement data. The processor may classify a current mental state based on the EEG sensors, the filtered data, the brain activity data, or a combination thereof. The processor may filter the brain activity data with the movement data to provide filtered data. The processor may compare brain activity to movement data. The processor may isolate the brain activity by excluding movement data from the brain activity data to generate filtered data. The processor may monitor the brain activity, the movement data, or both for patterns. The processor may monitor for jaw clicking, TMJ, or both. The processor may provide feedback. The feedback may be haptic feedback, a text message, a light, or a combination thereof. The feedback may indicate that mindfulness, relaxation, or both are needed in view of the current mental state. The processor may be a computer, a microprocessor, or both. The processor may be part of a phone, a computer, cloud based, or a combination thereof. The processor may generate a personalized score based upon the feedback, a current mental state, or both.

The personalized score may be charted over time, collected, monitored in real time, compared to previous personalized scores, provide feedback if a personalized score is trending to a predetermined mental condition, or a combination thereof. For example, a score of 1 may indicate a low stress level (e.g., relaxed) and a score of 100 may indicate a high stress level. For example, on an average day a user usually trends with a score of 40. If the user begins trending in the 50s the processor may indicate that a break is needed or that some other techniques may be needed. The daily average score may become a baseline score for that particular user. The processor may tailor notices for that particular user. For example, one user may have a baseline score of 35 and another user may have a baseline score of 50 and the needs of each may be different, thus, the processor may provide different feedback at different times for each user. The processor may provide feedback so that a user may take corrective action. The processor may encourage good habits, discourage bad habits, or both by providing feedback. For example, the wearable device may vibrate, send a website to the user providing calming techniques, provide feedback that the user is performing a repetitive movement, or a combination thereof.

The wearable device taught herein may include a process of monitoring brain activity and providing feedback to the user. The process may include monitoring brain activity with EEG sensors. The EEG sensors are spaced apart and located adjacent to an ear of the user. The process may include generating brain activity data from the monitored brain activity. The process may include monitoring movements of the user with the one or more movement sensors. The process may generate movement data from the monitored movements of the user. The process may classify a current mental state of the user based on the EEG sensor. The process may provide a personalized score to the user based on the current mental state. The process may isolate the brain activity or the brain activity data by excluding the movement data or artifacts caused by movement in the brain activity data. The process may generate a personalized score. The process may compare a current personalized score to previous personalized scores. The process may determine a current mental state of the user. The process may request that the user characterize a current mental state so that when the process identifies a pattern the processor may provide feedback to the user regarding a prior characterization. The classification may be based upon direct user interaction, a database of classifications, a score, or a combination thereof. The process may generate a baseline mental state. The baseline may be generated by tracking scores of the user. The process may provide feedback to the user regarding trends, ways to change a mental state, corrective action, or a combination thereof. The process may encourage good habits, discourage bad habits, or both.

Reference will now be made in detail to certain illustrative implementations, examples of which are illustrated in the accompanying drawings. Wherever possible, the same reference numbers will be used throughout the drawings to refer to the same or like items.

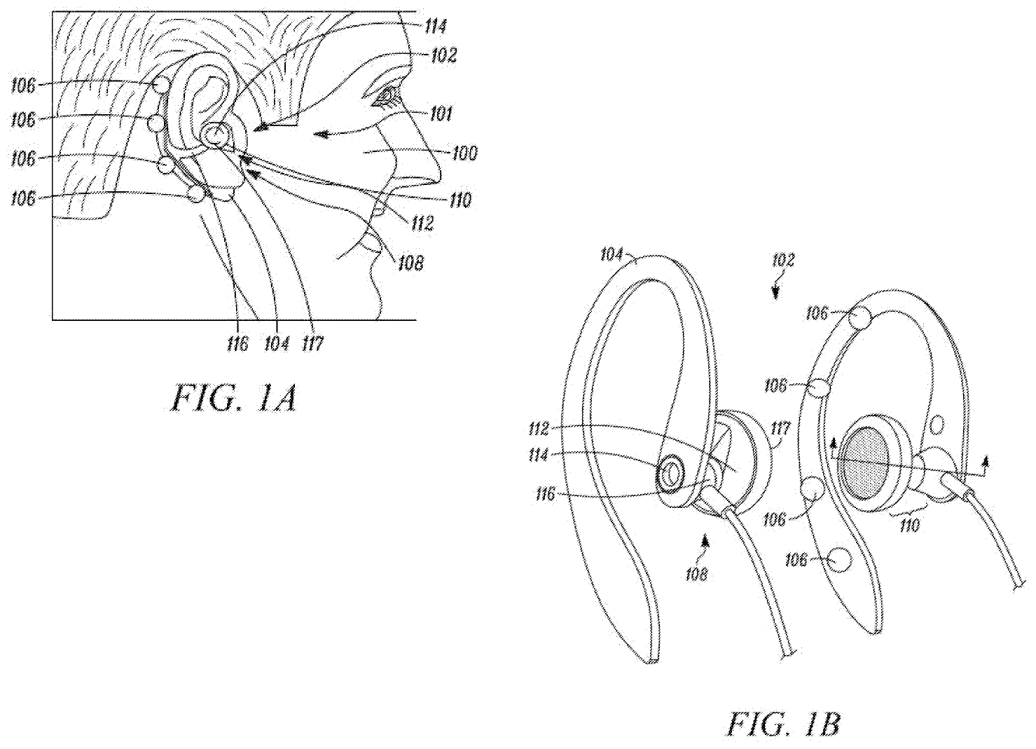

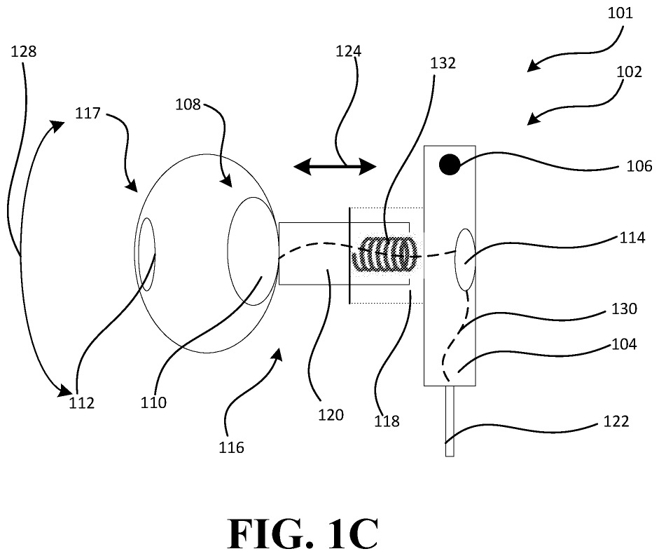

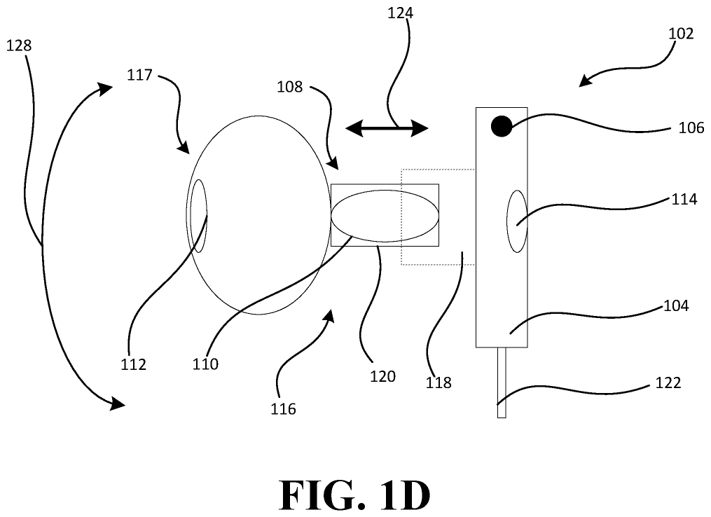

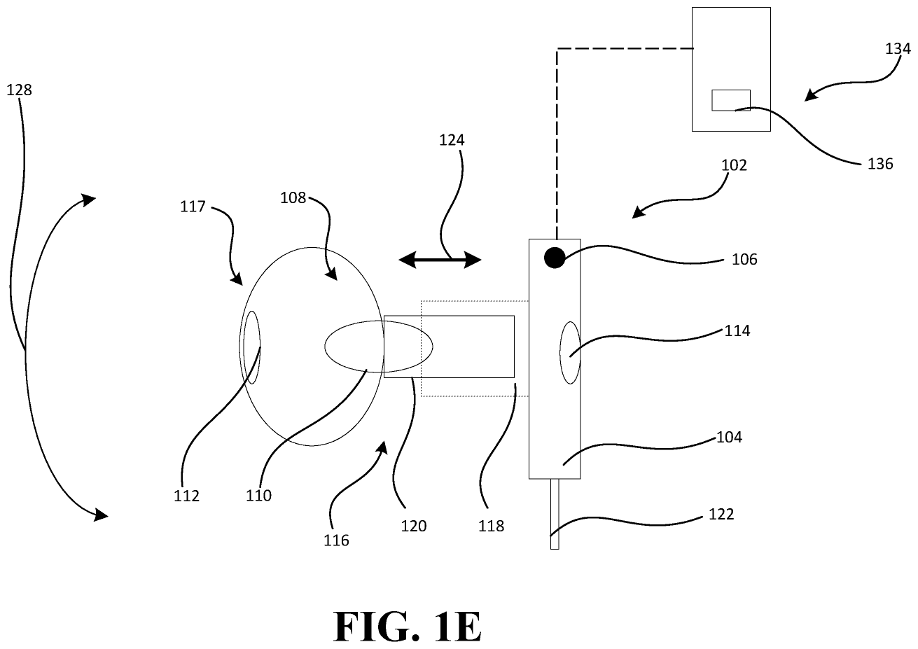

FIG. 1 illustrates a user 100 wearing a wearable device 101 shown as an ear device 102. The ear device 102 includes an ear loop 104 that connects the wearable device 101 to an ear of the user 100. The ear loop 104 extends around a portion of the user's 100 ear and assists in retaining the ear device 102 in place. The ear loop 104 hooks around the ear lobe to hold the ear lobe. The ear loop 104 includes electroencephalogram sensors (EEG sensors) 106 connected to and extending along the ear loop 104.

The EEG sensors 106 are spaced apart and extend around the ear. The ear loop 104 may include one or more, two or more, three or more, four or more, or five or more EEG sensors 106. The EEG sensors 106 may be located at a top region, bottom region, middle region, or a combination thereof of the ear loop 104. The EEG sensors 106 may be equally spaced apart. The EEG sensors 106 may be located behind the ear. The EEG sensors 106 may be in direct contact with skin of the user 100. The EEG sensors 106 may monitor brain activity of a user. The EEG sensors 106 may work in tandem to provide one brain activity wave. Each EEG sensor 106 may provide brain activity waves. The EEG sensors 106 may be used in tandem with movement sensors 108.

The movement sensors 108 may measure movements of the user 100. The movement sensors 108 may measure movement of the user's 100 face, ear, jaw, neck, or a combination thereof. The movement sensors 108 may be flexed, compressed, vibrated, or a combination thereof by the user 100. The movement sensors 108 may be an accelerometer, ultrasound, piezoelectric sensor (PES) 110, gyroscope, light sensor, optic cables, or a combination thereof. As shown, the movement sensors 108 are a PES 110.

The PES 110 functions to measure movements of the user 100. The PES 110 may be bent, flexed, compressed, moved, vibrated, or a combination thereof. The PES 110 may extend cantilever, be round, connected at a single end, or a combination thereof. The PES 110 may be located in the ear device 102 at a location proximate to a speaker 112 and a microphone 114.

The speaker 112 functions to provide sound to the user 100. The speaker 112 provides sound into an ear of the user 100. The speaker 112 may provide music, a phone conversation, or both. The speaker 112 may be provided in tandem with the microphone 114.

The microphone 114 functions to capture a voice of the user 100. The microphone 114 may be located opposite the speaker 112. The microphone 114 may permit the user 100 to use the ear device 102 to make a telephone call. The microphone 114 may extend outward away from an ear of the user 100. The microphone 114 may be located proximate to a support post 116.

The support post 116 functions to support the ear loop 104. The support post 116 is connected to the ear loop 104 at a first end and an ear tip 117 at a second end. The support post 116 may be flexible. The support post 116 may be rigid. The support post 116 may be straight, be curved, be “C” shaped, extend around a middle of an ear, extend over an ear, extend axially away from an ear, or a combination thereof. The support post 116 may be located proximate to the speaker 112, the microphone 114, or between the speaker 112 and the microphone 114.

FIG. 1B illustrates a pair of ear devices 102. One of the ear devices 102 extends into each ear. The ear devices 102 play music, connect to a telephone, provide sound, receive sound, or a combination thereof. The ear devices 102 each include an ear loop 104.

The ear loops 104 extend around an ear of the user 100 of FIG. 1A. The ear loops 106 extend from an ear canal, over the ear, and around the ear. The ear loops 106 may be made of or include a plastic, metal, a polymer, a rigid material, a flexible material, or a combination thereof. The ear loops 106 may be solid. The ear loops 106 may be hollow. Each ear loop 106 may include the EEG sensors 108. However, one ear loop 106 may be free of the EEG sensors 108 and one of the ear loops 106 may include the EEG sensors 108.

The EEG sensors 108 are spaced apart along the ear loop 106 and function to monitor and/or measure brain waves. The EEG sensors 108 may be located on an inside of the ear loops 106 so that the EEG sensors 108 are in direct contact with skin of the user. The EEG sensors 108 receive electrical signals from a user's brain to form brain wave patterns. The EEG sensors 108 are spaced apart along the ear loops 106. Each EEG sensor 108 may provide a wave form. The EEG sensors 108 may be combined to provide a single wave form. The EEG sensors 108 may be used in conjunction with movement sensors 108.

The movement sensors 108 function to measure movements of the user to generate movement data. The movement sensors 108 may monitor micro-movements of a user. The movement sensors 108 may monitor movements of a neck, head, face, ear, jaw, or a combination thereof. The movement sensors 108 may be any of the types of movement sensors 108 discussed herein. The movement data of the movement sensors 108 may be compared to the brain activity so that any anomalies or outliers generated by the movements of the user may be removed from the brain activity. The movement sensors 108 may be directly moved by the user. The movements sensors 108 may be indirectly moved via one or more components of the ear device 102 being moved that then move the movement sensors 108. The movement sensors 108 may include at least a piezoelectric sensor (PES) 100.

The PES 110 may be located between the speaker 112 and the microphone 114. The PES 110 may be located in a support post 116. The PES 110 may be the support post 116. The PES 110 may extend through the support post 116, be partially located within the support post 116, be partially located within the support post 116, or a combination thereof. The PES 110 may move with the support post 116.

The support post 116 may be axially movable, laterally movable, longitudinal movable, or a combination thereof. The support post 116 may be one solid piece that may be at least partially flexible, have flexible connections at one or both ends, or both. The support post 116 may axially extend out of a user's ear, from an ear tip 117 to the ear loop 104, or both.

The ear tip 117 functions to connect the ear device to a user, provide sound, house components, or a combination thereof. The ear tip 117 houses the microphone 114. The ear tip 117 may be malleable to fit within an ear canal, to support the ear device 102 within an ear canal, or both. The ear tip 102 may be adapted based upon a user. The ear tip 102 may be movable relative to the support post 116.

FIG. 1C illustrates a cross-sectional view of the wearable device 101, which as shown is an ear device 102. The ear device 102 includes the ear loop 104 connected to the ear tip 117 by the support post 116. The ear loop 104 includes the EEG sensors 106 and the microphone 114. The ear device 102 is powered by a power source 122. The power source 122 is a wired power source 122 that distributes the power by a wire 130.

The wire 130 functions to provide power, signals, or both to the components of the ear device 102. The wire 130 may be a copper wire, a fiber optic cable, or both. The wire 130 may extend into the ear loop 104, the support post 116, or the ear tip 117. The wire 130 may extend into the ear loop 104 through the support post 116 and into the ear tip 117.

The support post 116 may include a primary support post 118 and the secondary support post 120. The primary support post 118 and the secondary support post 120 may be movable relative one another. The primary support post 118 may receive all or a portion of the secondary support post 120 or vice versa. The primary support post 118 may axially move relative to the secondary support post 120 in a direction 124. The primary support post 118, the primary support post 120, or both may permit bending of the support post 116. The primary support post 118, the secondary support post 120, or both may be moved by a user and then biased back into a predetermined position by a bias member 132.

The bias member 132 may move the primary support post 118 relative to the secondary support post 120. The bias member 132 may bias by being compressed, being in tension, or a combination of both based on a force imparted on the bias member 132. The bias member 132 may be a spring, elastic, rubber, expandable, contractable, or a combination thereof. The bis member 132 may contact the PES 110, bias the secondary support post 120 into contact with the PES 110, bias the primary support post 118 into contact with the PES 110, or a combination thereof. The bias member 132 may bias the support post 116 so that movements of the ear tip 117 may be measured by the PES 110.

The ear tip 117 includes the movement sensor 108 and the speaker 112 to provide sound to the user. The ear tip 117 may be axially movable along the support post 116 in the direction 124 and a direction 128.

FIG. 1D illustrates an ear device 102 that is substantially identical to the ear device 102 of FIG. 1C. However, the movement sensor 108 shown as the PES 110 is located within the support post 116. As the primary support post 118 and the secondary support post 120 move relative to each other the PES 110 is moved so that the PES 110 senses the movements. The PES 110 may sense axial movement in the direction 124, rotational or bending movement in the direction 128, or both.

FIG. 1E illustrates an ear device 102 that is substantially identical to the ear device 102 of FIGS. 1C or 1D. As shown, the movement sensor 108 illustrated as the PES 110 is located partially within the support post 116 and partially within the ear tip 117. The PES 110 may measure axial movements of the support post 116 relative to the ear tip 117 in the direction 124 by the primary support post 118 moving relative to the secondary support post 120, movement of the support post 116 relative to the ear tip 117 in the direction 128, or both.

The ear device 102 is in communication (e.g., wired or wireless) with a display device 134 that includes a processor 136. Communications between the ear device 102 and the processor 136 can be transmitted from one to the other wirelessly (e.g., via Bluetooth, RF signal, Wi-Fi, near field communications, etc.) or through one or more wires 130. Although not depicted, the display device 134 and processor 136 may be used with the wearable devices 101 depicted in FIGS. 1A-1D. The display device 134 functions to display a mental state of the user, provide feedback, encourage good habits, discourage bad habits, provide a personalized score, provide haptic feedback, a text message, a light, make mindfulness suggestions, provide relaxation suggestions, or a combination thereof. The display device 134 may be part of or be a watch, a cell phone, a computer, a smart device, a tablet, or a combination thereof. The display device 134 may be part of a device that is connectable via a wireless connection such as Bluetooth or Wi-Fi or a wired connection via the power source 122 wire. The display device 136 may comprise a display screen for displaying images, characters, graphs, waveforms, or a combination thereof to at least one of the user or a medical professional. The display device 136 may further comprise one or more hard or soft buttons or switches configured to accept input by the user. The display device 136 may switch or be toggled between displaying user physiological information, feedback, current mental state, a personalized score, or a combination thereof.

The display device 134 may include an application, access a website, or both that are loaded on a processor 136 to process the brain waves, movement data, or both.

The processor 136 functions to collect brain activity data, collect movement data, and provide feedback to the user 100 of FIG. 1A. The processor 136 may isolate the brain activity by excluding movement data from the brain activity. The processor 136 may generate filtered data by removing the movement data from the brain activity data. The processor 136 may monitor patterns within the movement data, the brain activity data, or both. The processor 136 may analyze the movement data, the brain activity data, or both for patterns, behaviors related patterns, movement patterns, previously identified activity (e.g., a shape of a waveform), or a combination thereof. The processor 136 may gather, store, or both data from the EEG sensors 106, the movement sensors 108, or both.

In addition to the PES 110, the ear device 102 may further comprise additional movement sensors 108 such at least one of the as one or more accelerometers or gyroscopic components for determining whether and to what extent the user is in motion (i.e., whether the user is walking, jogging, running, swimming, sitting, or sleeping), breathing rhythm, breathing signals, or a combination thereof of a user. Information collected by at least one of the accelerometer(s) or gyroscopic components can also be used to calculate the number of steps a user has taken over a period of time. This activity information can also be used in conjunction with physiological information collected by other sensors (such as EEG sensors, heart rate, respiration rate, blood pressure, etc.) to determine a user's caloric expenditure, mental health, and other relevant information. The activity information may measure movements. The movements measured may be macro-movements such as walking or jogging. The movements may be micro-movements. The micro-movements may be caused by a surface of a user's skin being moved due to respiration, heartbeat, or a both. The micro-movements may have an amplitude (e.g., length) less than a predetermined amplitude in order for at least one of the accelerometer or gyroscope to at least one of the measure or record the micro-movements. For example, when a user walks the accelerometer may measure a movement of more than 1 cm, when the accelerometer detects a user heart beat the accelerometer may measure a movement of between 4 mm and 1 cm, and when the accelerometer measures a movement of 4 mm or less (e.g., a micro-movement). The micro-movements may be charted in wave form such that the micro-movements are charted with a peak and a valley. The amplitude of movement may assist the non-transitory computer readable medium or processor 136 in isolating movements caused by multiple sources (e.g., heart beat and respiration). The processor 136 may receive data from at least one of the PES 110, the accelerometer, or gyroscope related to movements of the user. The processor 136 may dynamically filter the data. The processor 136 may provide a respiratory signal regarding the respiration of the user. The processor 136 may analyze the data without regard to a position of the device relative to the user or a position of the user. The processor 136 may filter out unwanted signals and isolate only desired signals. For example, the processor 136 may learn which signals are of interest and the processor may analyze only those signals of interest. The processor 136 may be in communication with or include a non-transitory computer-readable medium.

The non-transitory computer-readable medium may store one or more programs. The programs may be executable by the processor. The program may be configured to dynamically filter, determine a mental status, provide a personalized score, a respiratory signal, provide a respiration signal, display an output, or a combination thereof. The program may control the processor to monitor the movement sensor 108 and the EEG sensor 106. The program may use the processor to perform the dynamic filtering discussed herein. The program may determine a mental health or a personalized score as discussed herein. The program may display an output as discussed herein on a device or a screen of a device as discussed herein. The program, the processor, or both may determine filter weights to apply to the movement data, the brain activity data, or both as discussed herein.

The processor 136 may be configured to continuously collect data from the EEG sensor 106, the movement sensor 108, or both. However, certain techniques can be employed to reduce power consumption and conserve battery life of the ear device 102. For instance, only one of the EEG sensor 106 or the movement sensor 108 may continuously collect information.

The ear device 102 may also determine the heart rate, respiratory rate, blood pressure, oxygen levels, and other parameters of a user involves collecting a signal indicative of blood flow pulses from a targeted area of the user's tissue. This information can be collected using, for example, a light source, a photo detector, or a pressure sensor. Some implementations may use multiple light sources and they may be of varying colors (e.g., green, blue, red, etc.). For example, one light source may be an IR light source and another might be an LED light (such as a red LED). Using both an IR light source and a colored LED light (such as red) can improve accuracy as red light is visible and most effective for use on the surface of the skin while IR light is invisible yet effective for penetration into the skin. Such implementations may comprise multiple photo detectors, one or more configured to detect colored LED light (such as red) and one or more configured to detect IR light. These photo detectors (for detecting light of different wavelengths) can be combined into a single photodiode or maintained separate from one another. Further, the one or more light sources and one or more photodetectors could reside in the ear device 102.

The physiological information from the EEG sensor 106, the movement sensor 108, or both may be graphically displayed or represented by a waveform on the display device 134. The graphical display may be provided as an output. The output may include physiological information of a user. For example, the information collected may be categorized and then graphically represented as an output or two or more outputs. The one or more outputs may be one or more waveforms, two or more waveforms, or three or more waveforms. The waveforms may be individually created. The waveforms may overlay one another. The waveforms may be created by categorizing the brain waves, the movements, or both. The waveforms may be a one or more waveforms such as a sine wave or a sinusoidal pattern. The output may have one graph having respiration signals, mental state, and a graph having a heart rate. The output may be words, numbers, or both. For example, the display device 134 may tell the user 100 to take a mental break of that their brain activity is escalating (e.g., stress level is raising).

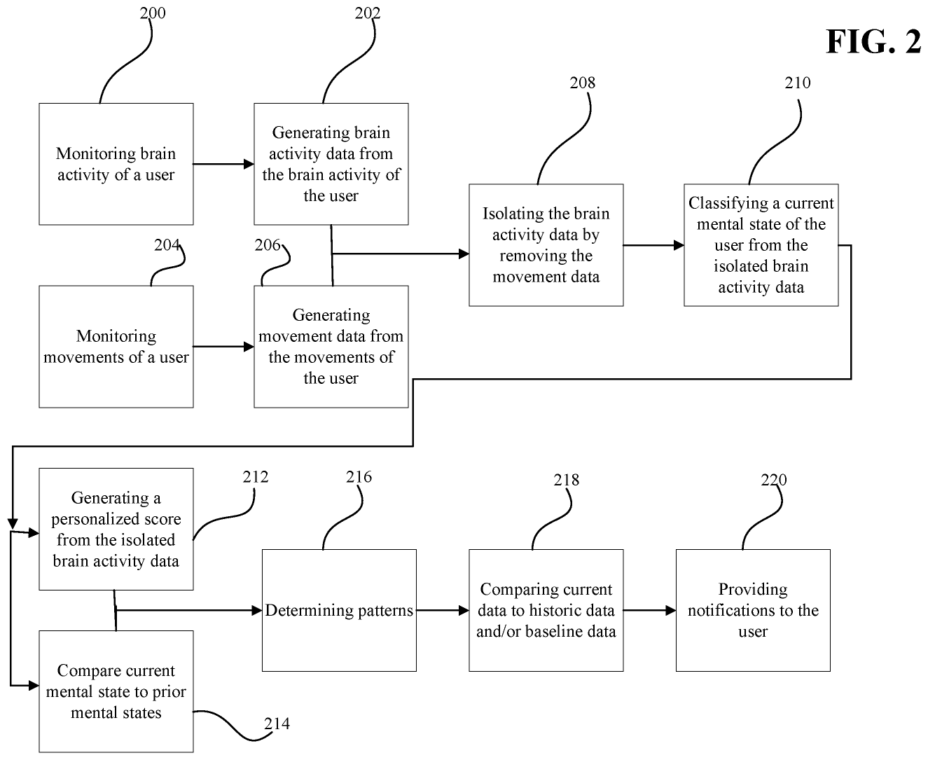

FIG. 2 illustrates a process diagram demonstrating process steps and/or steps that may be performed by the processor 136. The EEG sensors 106 monitor brain activity 200 of a user. The EEG sensors 106 may be placed proximate to a user's skin and preferably behind an ear of the user. The brain activity 200 is converted, generated, or saved as brain activity data 202. The brain activity data 202 may be charted, plotted, stored, charted over time, classified, categorized, or a combination thereof.

The wearable device 101 monitors movements of a user 204. The movements being monitored 204 may be micromovements that may impact the brain activity. The movements 204 may then be converted, generated, saved, or a combination thereof as movement data 206. The movement data 206 may be monitored for patterns, plotted, saved, charted over time, classified, categorized, or a combination thereof. For example, the movement data 206 may be separated into micro-movements and macro-movements.

The process, processor, or both may isolate the brain activity data from the movement data 208. For example, movements of the user may be detected by the brain activity sensors. Movements of the user may generate anomalies, blips, outliers, jumps in the brain activity data, or a combination thereof. The isolation 208 may compare the activity data and the movement data so that when movements are recorded the corresponding changes in the activity data may be removed. The isolation 208 may generated a filtered data or an isolated data.

The isolated data 208 may be classified 210. The classification 210 may generate a type of mental state currently being experienced by a user. The classification 210 may be stressed, calm, anxious, worried, bored, apathetic, or a combination thereof. The isolated data 208 may be classified 210 and scored 212 in series or in parallel.

The personalized score 212 may be plotted over time. The personalized score 212 may be on a scale of 1 to 100. The personalized score 212 may be generated in real time. The personalized score 212 may be charged in real time, provided to the user in real time, or a combination thereof. The personalized score 212 may be adjusted over time using computer learning so that the personalized score is tailored for an individual. The personalized score 212 may be compared to prior personalized scores, a baseline, or both.

The current mental state 214 may be compared to prior mental states, previously classified mental states, or both. The current mental state 214 may be classified by a user so that the processor may be trained to identify mental states for a particular user. For example, if a user is experiencing anxiety the user will provide the processor with an input that the mental state is anxiety. Thus, in future events the process, processor, or both may compare a current mental state 214 with prior mental states to provide the user with mental state, or a direction the current mental state is trending.

The process, processor, or both may monitor the brain activity, movements, or both to determine patterns or trends 216. For example, a user may move their jaw in a particular way when they are stressed and the monitored movements may indicate that this is an indication of stress. In another example, if a user becomes stressed during eating or has an eating disorder then the movement data may be able to notice that there is a trend that eating triggers a particular mental state. In determining patterns 216 stressor events may be identified. The step of determining patterns 216 may assist a user in avoiding triggers that cause a mental state that is uncomfortable to the user. The patterns 216 may be determined in real time and then compared to historic data and/or baseline data 218.

The process, processor, or both in comparing the current data (e.g., the current isolated brain activity data) may provide a higher level of confidence as to a current mental state, a personalized score, a classification of a mental state, or a combination thereof 218. The step of comparing 218 may include comparing graphs to graphs, scores to scores, trends to trends, patterns to patterns, or a combination thereof. The comparing 218 may look in a graph for a wave of a certain shape, wave with a certain amplitude, wave with a certain length, wave with certain distances between peaks or valleys, or a combination thereof. The comparing 218 may compare waves to baseline waves, numbers to baseline numbers, or both. The comparing 218 may be performed to validate the classification 210 before being provided to the user 220.

An output 220 may be provided to the user. The output 220 may be a number, a verbal description, a comparison to a baseline, a graphical representation, or a combination thereof. The output 220 may be provided on a display device 134. The output 220 may be provided on demand, intermittently, upon a mental state change, when a concerning mental state is beginning, during a triggering event, or a combination thereof. The output 220 may send feedback to a user, alert a user to change current status, provide a user with reminders, provide a user with encouragement, discourage a user from habits, provide a user with exercises to change a mental state, or a combination thereof.

It may be appreciated that various changes can be made therein without departing from the spirit and scope of the disclosure. Moreover, the various features of the implementations described herein are not mutually exclusive. Rather any feature of any implementation described herein may be incorporated into any other suitable implementation.

Additional features may also be incorporated into the described systems and methods to improve their functionality. For example, those skilled in the art will recognize that the disclosure can be practiced with a variety of physiological monitoring devices, including but not limited to heart rate and blood pressure monitors, and that various sensor components may be employed. The devices may or may not comprise one or more features to ensure they are water resistant or waterproof. Some implementations of the devices may hermetically sealed.

Other implementations of the aforementioned systems and methods will be apparent to those skilled in the art from consideration of the specification and practice of this disclosure. It is intended that the specification and the aforementioned examples and implementations be considered as illustrative only, with the true scope and spirit of the disclosure being indicated by the following claims.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A device comprising:
one or more ear loops configured to extend at least partially around an ear of a user, the one or more ear loops include:
three or more electroencephalogram sensors (EEG sensors) distributed along the one or more ear loops so that the three or more EEG sensors extend around the ear of the user;
a support post extending from the one or more ear loops; and
an ear tip connected to and supported by the support post;
one or more movement sensors configured to monitor movement of the user, wherein the one or more movement sensors include one or more pizeoelectric sensors (PES).

2. The device of claim 1, wherein the PES is located within the ear tip.

3. The device of claim 2, wherein the PES is in communication with the support post.

4. The device of claim 1, wherein the support post includes a primary support post and a secondary support post that are connected together and movable relative to each other.

5. The device of claim 1, wherein the three or more EEG sensors are spaced apart and are located on the one or more ear loops so that one of the EEG sensors is located at a top of the ear, a second of the EEG sensors is located at a middle of the ear, and a third of the EEG sensors is located at a bottom of the ear.

6. The device of claim 2, wherein the support post is rigid and the PES is flexible to measure movements of the user.

7. The device of claim 6, wherein the movements of the user the PES measures is jaw movement.

8. The device of claim 2, wherein a portion of the PES is located within the ear tip and a portion of the PES extends out of the ear tip.

9. A device comprising:
two or more sensors comprising:
two or more electroencephalogram sensors (EEG sensors) configured to monitor brain activity of a user; and
one or more movement sensors configured to monitor movement of the user, wherein the one or more movement sensors include one or more piezoelectric sensors (PES); and
a processor configured to:
detect the brain activity of the user with the two or more EEG sensors;
detect movements of the user with the one or more PES to generate movement data;
isolate the brain activity by excluding the movement data from the brain activity to generate filtered data, and
provide feedback to the user regarding a current mental state of the user based upon the filtered data.

10. The device of claim 9, wherein the processor is configured to monitor patterns in the movement data to provide the user with an output regarding behavior.

11. The device of claim 10, wherein the PES detects movement of a jaw of the user.

12. The device of claim 9, wherein the movement data includes jaw clicking that is measured by the PES and the jaw clicking is removed from the brain activity.

13. The device of claim 9, wherein the feedback is haptic feedback, a text message, a light, or a combination thereof indicating that mindfulness, relaxation, or both are needed in view of the current mental state.

14. A method comprising:
monitoring brain activity of a user with two or more electroencephalogram sensors (EEG sensors) that are spaced apart and located adjacent to an ear of the user;
generating brain activity data from the monitored brain activity;
monitoring movements of the user with one or more movement sensors that include one or more piezoelectric sensors (PES);
generating movement data from the monitored movements of the user;
classifying a current mental state of the user based on the EEG sensors; and
providing a personalized score to the user based on the current mental state.

15. The method of claim 14, further comprising isolating the brain activity data by excluding the movement data to generate filtered data.

16. The method of claim 14, further comprising comparing a current personalized score to prior personalized scores to determine the current mental state of the user.

17. The method of claim 16, wherein the current mental state is classified and provided to the user.

18. The method of claim 14, further comprising generating a baseline mental state by tracking the personalized scores of the user.

19. The method of claim 18, further comprising providing trend feedback to the user based upon the movement data so that a user can take corrective action to reduce trends.

20. The method of claim 18, further comprising providing feedback to encourage good habits and discourage bad habits.
